(19) United States
(12) Patent Application Publication
(10) Pub. No.: US 2011/0118728 A1
(43) Pub. Date: May 19, 2011

(54) CONTROL OF HIGH-INTENSITY PULSED ELECTRICAL FIELDS IN SURGICAL APPLICATIONS

(75) Inventors: Tammo Heeren, Aliso Viejo, CA (US); John C. Huculak, Mission Viejo, CA (US); Steven W. Kovalcheck, Aliso Viejo, CA (US)

(73) Assignee: ALCON RESEARCH, LTD., Fort Worth, TX (US)

(21) Appl. No.: 12/618,244

(22) Filed: Nov. 13, 2009

(51) Int. Cl. A61F 9/007; A61B 18/12

(52) U.S. Cl. 606/34

(57) ABSTRACT

An eye surgery apparatus includes a HIPEF probe comprising at least two electrodes and is configured for delivery of a high-intensity pulsed electrical field to a surgical site within an eye via the electrodes. Embodiments also include a transducer configured to monitor one or more surgical parameters within the eye during application of the high-intensity pulsed electrical field to the surgical site, a pulse generation circuit configured to generate a series of electrical pulses for application to the electrodes to create the high-intensity pulsed electrical field, and a control circuit, operatively connected to the at least one transducer and the pulse generation circuit and configured to automatically adjust one or more characteristics of the series of electrical pulses, based on the one or more monitored surgical parameters. With these apparatus, the amount of energy delivered can be limited to levels necessary for effective operation without over-exposing the vitreous.

### TECHNICAL FIELD

The present invention relates generally to the field of eye surgery and more particularly to methods and apparatus for performing eye surgery using high-intensity pulsed electric fields.

### BACKGROUND

Techniques and apparatus for dissociation and removal of highly hydrated macroscopic volumes of proteinaceous tissue from the human eye have been previously disclosed. In particular, techniques for dissociation and removal of highly hydrated macroscopic volumes of proteinaceous tissue using rapid variable direction energy field flow fractionation have been disclosed by Steven W. Kovalcheck in “System For Dissociation and Removal of Proteinaceous Tissue”, U.S. patent application Ser. No. 11/608,877, filed 11 Dec. 2006 and published 5 Jul. 2007 as U.S. Patent Application Publ. No. 2007/0156129 (hereinafter “the Kovalcheck application”), the entire contents of which are incorporated herein by reference.

The techniques disclosed in the Kovalcheck application were described in detail in terms of vitreoretinal surgery. However, those of ordinary skill in the art will readily understand that those techniques are applicable to medical procedures in other areas in the body of humans or animals. As explained in the Kovalcheck application, prior art procedures for vitreoretinal posterior surgery have relied for decades on mechanical or traction methods such as: 1) tissue removal with shear cutting probes (utilizing either a reciprocating or rotary cutter); 2) membrane transection using scissors, a blade, or vitreous cutters; 3) membrane peeling with forceps and picks; and 4) membrane separation with forceps and viscous fluids. While improvements in mechanisms, materials, quality, manufacturability, system support, and efficacy have progressed, many of the significant advancements in posterior intraocular surgical outcomes have been primarily attributable to the knowledge, fortitude, skill, and dexterity of the operating ophthalmic physicians.

However, the Kovalcheck application disclosed novel apparatus and methods for delivering a variable direction, pulsed high-intensity and ultra-short duration disruptive electric field (low energy) at a pulse duration, repetition rate, pulse pattern, and pulse train length tuned to the properties of the components of the intraocular extra-cellular matrix (ECM) to create tissue dissociation. In particular, the Kovalcheck application described a probe for delivering the pulsed rapid disruptive energy field to soft proteinaceous tissue surrounded by the probe. Once the adhesive mechanism between tissue constituents are compromised, fluidic techniques may be used to remove the dissociated tissue.

### SUMMARY

As described more fully below, embodiments of the present invention include an eye surgery apparatus that includes a HIPEF probe comprising at least two electrodes and configured for delivery of a high-intensity pulsed electrical field to a surgical site within an eye via the electrodes. These embodiments also include a transducer configured to monitor one or more surgical parameters within the eye during application of the high-intensity pulsed electrical field to the surgical site, a pulse generation circuit configured to generate a series of electrical pulses for application to the electrodes to create the high-intensity pulsed electrical field, and a control circuit, operatively connected to the at least one transducer and the pulse generation circuit and configured to automatically adjust one or more characteristics of the series of electrical pulses, based on the one or more monitored surgical parameters. With these apparatus, characteristics of the HIPEF pulses applied to the surgical site are automatically adjusted, based on the monitoring of one or more surgical parameters within the eye during the application of the high-intensity pulsed electrical field. In this manner, the amount of energy delivered, and the profile of the energy delivery, can be limited to levels necessary for effective operation without over-exposing the vitreous.

In various embodiments, the transducer may be a flow rate sensor configured to measure an aspiration flow rate, a pressure sensor configured to measure aspiration vacuum pressure, a bubble detection circuit configured to detect bubble formation at or near the surgical site, a temperature sensor configured to measure the temperature at or near the surgical site, and/or sensors configured to measure tissue properties such as pH, resistivity and conductivity at or near the surgical site. Of course, more than one of these transducers may be included in some embodiments.

In some embodiments, the pulse generation circuit is controlled to automatically adjust at least one electrical parameter selected from the group consisting of: a pulse frequency for at least one burst of the electrical pulses; a pulse duty cycle for at least one burst of electrical pulses; a burst repetition rate for two or more bursts of electrical pulses; a pulse amplitude for one or more of the electrical pulses; a pulse duration for one or more of the electrical pulses; a pulse rise-time for one or more of the electrical pulses; a pulse fall-time for one or more of the electrical pulses; and a pulse shape for one or more of the electrical pulses. In some embodiments, the pulse generation circuit may be controlled to automatically adjust an activation sequence of the electrodes for at least one burst of the electrical pulses, in response to the monitoring of the one or more surgical parameters. In some of these or other embodiments, pulse characteristics may be adjusted based further on an operator-selected surgical treatment type, an operator-selected surgical treatment location, an elapsed surgical treatment time, or a cumulative metric indicative of energy delivered to the surgical site, or some combination thereof.

Methods for controlling application of high-intensity pulsed electric field (HIPEF) energy during eye surgery are also disclosed. An exemplary method comprises applying a high-intensity pulsed electrical field to a surgical site within an eye, using a high-intensity pulsed-electrical-field (HIPEF) probe comprising at least two electrodes, monitoring one or more surgical parameters within the eye, during said application of the high-intensity pulsed electrical field, and automatically adjusting one or more characteristics of a series of electrical pulses applied to the electrodes to create the high-intensity pulsed electrical field, based on the one or more monitored surgical parameters. Other methods corresponding to the various apparatus summarized above are also disclosed.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary probe used for intraocular posterior surgery.

FIG. 2 is an enlarged perspective view of the tip of the probe of FIG. 1.

FIG. 3 is a schematic diagram of high-intensity pulsed electric field (HIPEF) vitrectomy apparatus according to some embodiments of the invention.

FIG. 4 is a process flow diagram illustrating an exemplary method for controlling application of HIPEF energy during eye surgery.

FIG. 5 illustrates pulse frequency control as a function of aspiration flow.

FIG. 6 illustrates pulse frequency control as a function of aspiration vacuum pressure.

FIGS. 7A and 7B illustrate pulse frequency control as a function of both aspiration flow and aspiration vacuum pressure.

### DETAILED DESCRIPTION

The present disclosure describes an apparatus and method for the dissociation and removal of highly hydrated macroscopic volumes of proteinaceous tissues, such as vitreous and intraocular tissue, during vitreoretinal surgery. More particularly, the techniques disclosed below are directed to methods and apparatus for detecting whether a high-intensity pulsed electric field (HIPEF) probe used for such surgery is actually positioned in an eye, before enabling the application of pulsed energy to the surgical site. Although the techniques disclosed herein are described in detail in terms of instruments and methods for traction-free removal of vitreous and intraocular membranes from the posterior region of the eye, without damaging the ultra-fine structure and function of the adjacent or adherent retina, those of ordinary skill in the art will understand the applicability of the disclosed invention for other medical procedures on both humans and animals.

As mentioned above, the Kovalcheck application (U.S. patent application Ser. No. 11/608,877) described a new approach to performing vitreoretinal surgery, using a high-intensity, short, directionally changing electrical field, rather than the classical mechanical means historically used to engage, decompose, and remove vitreous and intraocular tissues. The Kovalcheck application was based on the discovery that a transient change in tissue condition caused by the application of a ultra-short-high-intensity directionally changing electrical field is satisfactory for removal of macroscopic volumes of proteinaceous tissue. The technical success of mechanical and liquefying means supports the contention that vitreous material need not be obliterated or disrupted on a molecular level to be removed—rather, an innocuous macroscopic change of state is all that is needed for tissue removal. Accordingly, the removal of intraocular tissue enabled by the techniques described in the Kovalcheck application is traction-free.

The apparatus and method disclosed in the Kovalcheck application cause a local decoupling of the adhesive and structural relations in components of intraocular proteinaceous tissue, through the application of a rapidly changing electrical field. This localized dissociation of the adhesive and structural relations between components of intraocular proteinaceous tissue enables tractionless detachment between intraocular tissue components and the retinal membrane. Fluidic techniques (irrigation and aspiration) may be utilized during the tissue dissociation process to enhance the formation of a high-intensity ultra-short-pulsed electrical field and to remove disrupted tissue at the moment of dissociation. In general, it is intended that only the material within the applied high-intensity ultra-short-pulsed electrical field (also denoted high-intensity pulsed electric field, or HIPEF, herein) is assaulted and removed. Therefore, because only the material assaulted by the applied ultra-short pulses receives the high-intensity ultra-short-pulsed electrical field, there is no far-field effect during the tissue extraction process. This high-intensity ultrashort-pulsed electrical field assault leads to dissociation of the entrained macroscopic volume of intraocular proteinaceous tissue, and then aspiration removes the dissociated entrained macroscopic volume of tissue.

Generally speaking, then, a probe with two or more electrodes is inserted into the target hydrated tissue, vitreous or intraocular tissue. The ends of the electrodes are exposed at the distal end of the probe. An electrical pulse is transmitted down at least one of the electrodes while the other one or more electrodes act as the return conductors. A non-plasma electrical field is created between the electrodes. With each electric pulse, the direction of the created electrical field is changed by reversing polarity of the electric pulse, by electrode switching, or by a combination of both. Pulses may be grouped into bursts, which may be repeated at different frequencies and/or different amplitudes. Such pulse groups may be directed at heterogeneous tissue. The electrical pulse amplitude, duration, duty cycle and repetition rate along with continual changing of field direction, create the disruptive electrical field created across the orifice of the aspiration lumen. Tissue is drawn into the orifice of the aspiration lumen by fluidic techniques (aspiration). The tissue is then mixed or diluted with irrigation fluid and disassociated as it traverses the high-intensity ultra-short-pulsed directionally changing electric field. During a given interval, disorder is created in the entrained tissue by changing the direction of the electrical field between one or more of the electrodes at the tip of the probe.

The affected medium between the electrode terminations at the end of the probe consists of a mix of target tissue (e.g. vitreous) and supplemental fluid (irrigation fluid). The electrical impedance of this target medium in which the electrical field is created is maintained by the controlled delivery of supplemental fluid (irrigation fluid). In some embodiments, the supplemental fluid providing the electrical impedance is a conductive saline. The supplemental fluid may be provided by an irrigation source external to the probe, through one or more lumens within the probe or a combination of both. The supplemental may have properties (e.g., pH) and ingredients (e.g., surfactants) that may be conducive to protein dissociation.

The properties of the generated electrical energy field within the target medium are important. In the techniques disclosed in the Kovalcheck application and expanded upon herein, high-intensity, ultrashort pulses (sub-microseconds) of electrical energy are used. Tissue impedance, conductivity and dilution are maintained in the target medium by supplemental fluid irrigation, in some embodiments. The pulse shape, the pulse repetition rate, and the pulse train length may be tuned to the properties of the intraocular tissues, in some embodiments. In some embodiments, multiple pulse patterns may be employed to address the heterogeneity of intraocular tissue.

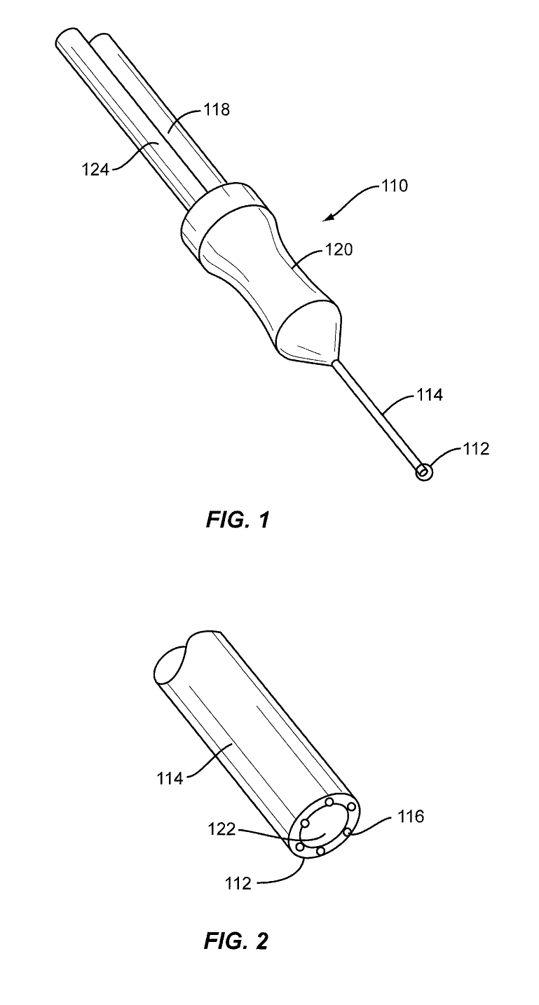

One application of the system described herein is for the treatment of pathologic retinal conditions. An exemplary apparatus for this treatment is shown in FIG. 1, which illustrates a HIPEF probe 110 comprising a hollow probe shaft 114 extending from handle 120 to probe shaft tip 112, an aspiration line 118, and electrical cable/transmission line 124. FIG. 2 illustrates details of the probe shaft 114 and probe shaft tip 112; a plurality of electrodes 116, connected to electrical cable 124, are exposed at the tip 112, and surround an aspiration lumen 122 providing an aspiration pathway to aspiration tube 118.

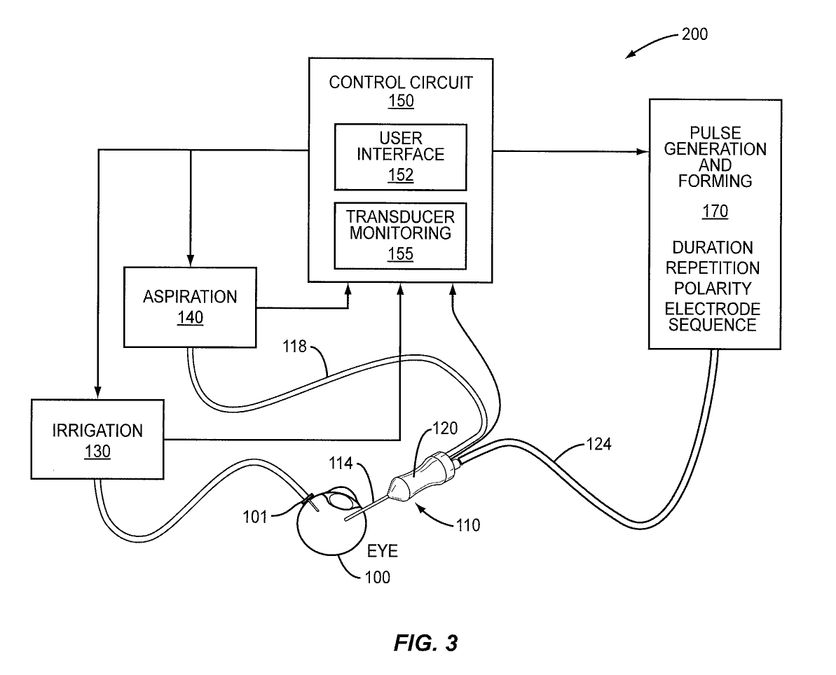

The tip 112 of probe 110 may be inserted by a surgeon into the posterior region of an eye 100 via a pars plana approach 101, as shown in FIG. 3, using handle 120. Using a standard visualization process, vitreous and/or intraocular membranes and tissues are engaged by the tip 112 at the distal end of the hollow probe shaft 114. Irrigation 130 and aspiration 140 mechanisms are activated, by control circuit 150, and ultra-short high-intensity pulsed electric energy from a high voltage pulse generator 170 (which includes a pulse-forming network and switching circuit, in some embodiments) are sent to the tip 112 via cable 124 (which may comprise a transmission line, for example), creating a disruptive high-intensity ultra-short-pulsed electrical field within the entrained volume of tissue. The adhesive mechanisms of the entrained constituents of the tissue that are drawn toward the probe tip 112 via aspiration through an aspiration line 118 connected to an aspiration lumen 122 in the hollow probe shaft 114 are dissociated, and disrupted tissue removed with the aid of the employed fluidic techniques. Engagement may be axial to or lateral to the tip 112 of the hollow probe shaft 114; extracted tissue is removed through the aspiration lumen 122 via a saline aspiration carrier to a collection module.

In some embodiments, the apparatus pictured in FIGS. 1 to 3 delivers high-intensity pulsed electric fields (HIPEF) at a pulse duration, repetition rate, pulse pattern, and pulse train length tuned to the properties of the components of the intraocular extra-cellular matrix. The pulse power generator 170 for the system 200 pictured in FIG. 3 delivers pulsed DC or gated AC against a low impedance of vitreous and the irrigating solution. Included in the system are energy storage, pulse shaping, transmission, and load-matching components. In some embodiments, the peak output voltage of the high voltage generator 170 is sufficient to deliver up to a 300 kV/cm field strength using the electrodes 116 at the distal end 112 of the hollow surgical probe shaft 114 (see FIG. 2). Generally, the pulse duration is short relative to the dielectric relaxation time of protein complexes. Further, the pulse duration, repetition rate, and pulse train length (i.e., duty cycle) are chosen to avoid the development of thermal effects (“cold” process). Thus, in some embodiments the system 200 generates and delivers square-shaped or trapezoidal-shaped pulses with rise times, fall times and pulse durations in the nanosecond range, and with voltages produced by the pulse forming network within generator 170 of greater than one kilovolt and in some cases in the tens of kilovolts.

Pulse generator 170 is configured to control pulse duration and repetition rate, and in some embodiments is configured to generate a stepwise continual change in the direction of the electrical field by switching between electrodes, reversing polarity between electrodes or a combination of both in an array of electrodes at the tip 112 of the hollow probe shaft 114. This continual change in the direction of the electrical field creates disorder in the entrained tissue without causing dielectric breakdown of the carrier fluid between the electrodes or thermal effects.

In various applications, the apparatus and techniques described herein may be applied to remove all of the posterior vitreous tissue, or specific detachments of vitreous tissue from the retina or other intraocular tissues or membranes. Engagement, disruption and removal of vitreous tissue, vitreoretinal membranes, and fibrovascular membranes from the posterior cavity of the eye and surfaces of the retina are critical processes pursued by vitreoretinal specialists, in order to surgically treat sight-threatening conditions such as diabetic retinopathy, retinal detachment, proliferative vitreoretinopathy, traction modalities, penetrating trauma, epi-macular membranes, and other retinopathologies. Though generally intended for posterior intraocular surgery involving the vitreous and retina, it can be appreciated that the techniques described herein are applicable to anterior ophthalmic treatments as well, including traction reduction (partial vitrectomy); micelle adhesion reduction; trabecular meshwork disruption, manipulation, reorganization, and/or stimulation; trabeculoplasty to treat chronic glaucoma; Schlemm's Canal manipulation, removal of residual lens epithelium, and removal of tissue trailers. Applicability of the disclosed apparatus and methods to other medical treatments will become obvious to one skilled in the art, after a thorough review of the present disclosure and the attached figures.

As noted above, one or more characteristics of the series of electrical pulses applied to the surgical site within the eye may be tuned to the properties of the intraocular tissues, in some embodiments. In some cases, multiple pulse patterns may be employed to address the heterogeneity of intraocular tissue. Characteristics that may be tuned include a pulse shape, a pulse repetition rate, and a pulse train length. Other characteristics applicable to one or more bursts of electrical pulses, any of which might be tuned, include, but are not limited to: a pulse frequency for at least one burst of electrical pulses, a pulse duty cycle for at least one burst of electrical pulses, a burst repetition rate for two or more bursts of electrical pulses, a pulse amplitude for one or more electrical pulses, a pulse duration for one or more electrical pulses, a pulse rise-time for one or more electrical pulses, a pulse fall-time for one or more electrical pulses, and a pulse shape for one or more of the electrical pulses.

In various embodiments of the present invention, one or more of these characteristics is automatically adjusted, based on the monitoring of one or more surgical parameters within the eye during the application of the high-intensity pulsed electrical field. In this manner, the amount of energy delivered, and the profile of the energy delivery, can be limited to levels necessary for effective operation without over-exposing the vitreous.

Relevant surgical parameters include, but are not limited to an irrigation or aspiration flow rate, aspiration vacuum pressure, temperature at or near the surgical site, the presence of bubble formation at the surgical site or tissue properties such as pH, resistivity or conductivity at or near the surgical site. Other factors that may be considered in adjusting the pulse characteristics include the treatment location, the type of treatment, the cumulative or averaged amount of delivered power/energy, the cumulative treatment time, and the like. In some cases, one or more of these latter factors may be used to determine limits for the variation based on the measured surgical parameters. For example, a treatment type and treatment location selected by the operator may be used by the control circuit 150 to establish boundary conditions for the burst parameters, with the controller 150 subsequently adjusting the pulse parameters within this bounded space, based on the monitored surgical parameters, to provide optimum efficiency.

Several of the surgical parameters mentioned above may be monitored using an appropriate transducer, corresponding to the measured parameter, such as a flow rate sensor, a pressure sensor, a thermal sensor, a bubble formation detector, pH sensor, resistance detector and so on. In any given embodiment, one or more of these transducers may be completely or partly located within the probe 110; in others (e.g., an aspiration flow sensor), the transducer may be located elsewhere. In any case, one or more of such transducers are monitored, e.g., using the transducer monitor section 155 of control circuit 150, with the results of the monitoring used to control one or more of the pulse characteristics discussed earlier.

More particularly, an eye surgery apparatus according to some embodiments of the invention may comprise, in addition to a multi-electrode HIPEF probe configured to deliver a high-intensity pulsed electrical field to a surgical site in an eye and a pulse generation circuit configured to generate a series of electrical pulses for application to the electrodes: at least one transducer configured to monitor one or more surgical parameters within the eye during application of the high-intensity pulsed electrical field to the surgical site; and a control circuit, operatively connected to the at least one transducer and the pulse generation circuit and configured to automatically adjust one or more characteristics of the series of electrical pulses, based on the one or more monitored surgical parameters. In some embodiments, this is done by providing a control signal to a pulse generation circuit that is configured to selectively respond to the control signal by adjusting at least one electrical parameter selected from the group comprising: a pulse frequency for at least one burst of the electrical pulses; a pulse duty cycle for at least one burst of electrical pulses; a burst repetition rate for two or more bursts of electrical pulses; a pulse amplitude for one or more of the electrical pulses; a pulse duration for one or more of the electrical pulses; a pulse rise-time for one or more of the electrical pulses; a pulse fall-time for one or more of the electrical pulses; and a pulse shape for one or more of the electrical pulses.

In some embodiments, the control circuit automatically adjusts an activation sequence for the electrodes of the multi-electrode HIPEF probe—in these embodiments, the two or more electrodes of the HIPEF probe are configured to be selectively activated by the pulse generator circuit for each of the electrical pulses, and the control circuit is configured to automatically adjust one or more characteristics of the series of electrical pulses by providing a control signal to the pulse generation circuit, the control signal directing the pulse generation circuit to adjust an activation sequence of the electrodes for at least one burst of the electrical pulses.

In various embodiments, the adjustment of one or more characteristics of the series of electrical pulses applied to the surgical site is based on one or more factors in addition to the surgical parameters measured by the transducer(s). For instance, in some embodiments, the control circuit is configured to automatically adjust one or more characteristics of the series of electrical pulses based further on an operator-selected surgical treatment type. (In some embodiments of the system 200 pictured in FIG. 3, the operator selects the treatment type using the user interface 152.) In these and other embodiments, the control circuit may be further configured to automatically adjust one or more characteristics of the series of electrical pulses based further on an elapsed surgical treatment time, and/or based on a cumulative metric indicative of energy delivered to the surgical site.

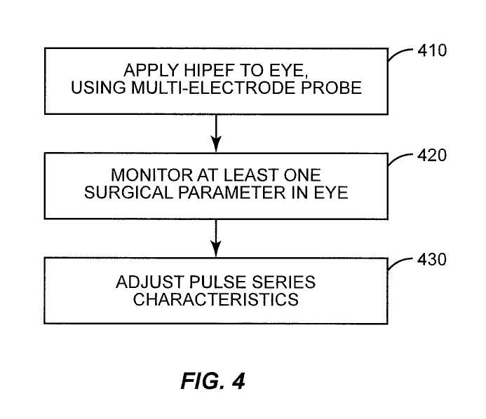

Given the various examples of apparatus discussed above, those skilled in the art will appreciate that FIG. 4 is a process flow diagram illustrating a generalized method for controlling application of high-intensity pulsed electric field (HIPEF) energy during eye surgery, such as might be implemented with one of the systems described above. The illustrated process begins, as shown at block 410, with the application of a high-intensity pulsed electrical field to a surgical site within an eye, using a HIPEF probe that comprises at least two electrodes. As shown at block 420, one or more surgical parameters within the eye are monitored, during the application of the high-intensity pulsed electrical field. This monitoring may comprise direct measurement of physical parameters within the eye, such as temperature or pH, or may comprise indirect measurement. Examples of the latter include measurements of aspiration flow rate or irrigation flow rate, which may be based on observations of fluid flow at an irrigation source or at an aspiration fluid reservoir. In any event, as shown at block 430, the process continues with the automatic adjusting of one or more characteristics of a series of electrical pulses applied to the electrodes to create the high-intensity pulsed electrical field, based on the one or more monitored surgical parameters.

Examples of the types of surgical parameters that may be monitored, as well as of the pulse characteristics that can be adjusted, have already been given above. Those skilled in the art will appreciate that these are illustrative, and not limiting, examples. Those skilled in the art will thus appreciate that various embodiments of the method pictured in FIG. 4 may include monitoring an aspiration flow rate, measuring aspiration vacuum pressure, detecting bubble formation at or near the surgical site, and/or measuring temperature at or near the surgical site and measuring tissue properties such as pH, resistivity or conductivity at or near the surgical site. Likewise, various embodiments of the illustrated method may comprise adjusting at least one electrical parameter selected from the group consisting of: a pulse frequency for at least one burst of electrical pulses, a pulse duty cycle for at least one burst of electrical pulses, a burst repetition rate for two or more bursts of electrical pulses, a pulse amplitude for one or more electrical pulses, a pulse duration for one or more electrical pulses, a pulse rise-time for one or more electrical pulses, a pulse fall-time for one or more electrical pulses; and a pulse shape for one or more electrical pulses.

Further, in some embodiments, adjusting one or more characteristics of the series of electrical pulses may comprise adjusting an activation sequence of the electrodes for at least one burst of the electrical pulses; in these and other embodiments, automatically adjusting one or more characteristics of the series of electrical pulses may be further based on one or more of: an operator-selected surgical treatment type, an operator-selected surgical treatment location, an elapsed surgical treatment time, or a cumulative metric indicative of energy delivered to the surgical site.

Some desired relationships between monitored surgical parameters and the controlled pulse characteristics will be apparent to those of ordinary skill in the art. For instance, an increase in vitreous temperature above a certain pre-determined level might indicate that one of a pulse frequency, pulse duty cycle, burst repetition rate, or pulse amplitude should be reduced, to avoid the delivery of damaging energy to the surgical site. Other desired relationships may be less apparent, but can be developed empirically. In either case, a pulse generation control circuit, such as the control circuit 150 of FIG. 3 can be pre-configured, e.g., with appropriate software and/or firmware instructions, to carry out adjustments of one or more pulse characteristics according to the appropriate relationship.

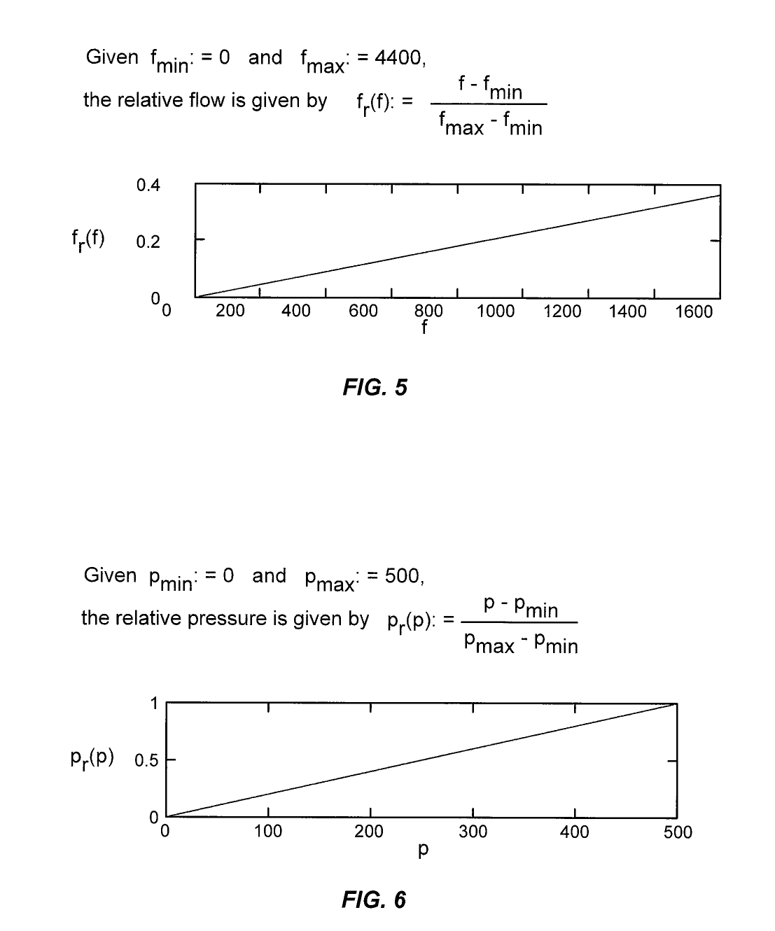

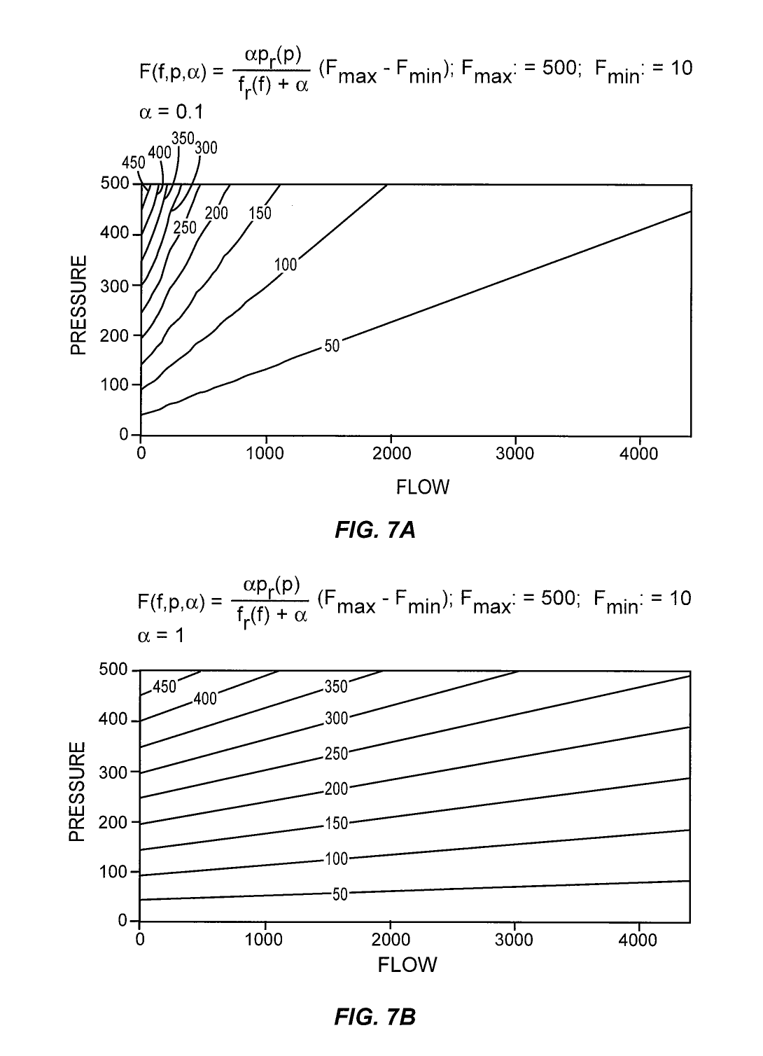

Examples of such a relationship are illustrated in FIGS. 5 and 6, which illustrate functions for a relative aspiration flow rate and relative aspiration vacuum pressure respectively. In some embodiments of the invention, the relative aspiration flow rate and/or relative aspiration vacuum pressure may be used as scaling factors to adjust a nominal pulse frequency rate. Various transfer functions may be implemented in some embodiments of the invention, to derive adjustments to pulse frequency as a function of monitored flow or pressure. Of course, other transfer functions are possible, including functions that map similar monitored parameters to other pulse characteristics, as are functions that relate several monitored parameters to one or more pulse characteristics. One example of the latter is illustrated in FIGS. 7A and 7B, which illustrate a family of transfer functions relating both the relative flow and relative pressure terms of FIGS. 5 and 6 to pulse frequency, with an adjustment parameter a adjusting the balance between flow and pressure. In FIG. 7A, the adjustment parameter a is 0.1, resulting in a pulse frequency that is a relatively strong function of the flow rate. In FIG. 7B, the adjustment parameter a is 1, resulting in a pulse frequency that is only slightly dependent on flow rate, but strongly dependent on aspiration vacuum pressure. Of course, those skilled in the art will recognize that similar transfer functions, and transfer functions involving other parameters and/or pulse characteristics are possible.

Indeed, all of the preceding descriptions of various methods and apparatus for controlling the application of high-intensity pulsed electric field energy during eye surgery were given for purposes of illustration and example, and those skilled in the art will appreciate that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A method of performing eye surgery, the method comprising:
applying a high-intensity pulsed electrical field to a surgical site within an eye, using a high-intensity pulsed-electrical-field (HIPEF) probe comprising at least two electrodes;
monitoring one or more surgical parameters within the eye, during said application of the high-intensity pulsed electrical field; and
automatically adjusting one or more characteristics of a series of electrical pulses applied to the electrodes to create the high-intensity pulsed electrical field, based on the one or more monitored surgical parameters.

2. The method of claim 1, wherein monitoring one or more surgical parameters within the eye comprises monitoring aspiration flow rate.

3. The method of claim 1, wherein monitoring one or more surgical parameters within the eye comprises measuring aspiration vacuum pressure.

4. The method of claim 1, wherein monitoring one or more surgical parameters within the eye comprises detecting bubble formation at or near the surgical site.

5. The method of claim 1, wherein monitoring one or more surgical parameters within the eye comprises measuring a temperature at or near the surgical site.

6. The method of claim 1, wherein monitoring one or more surgical parameters within the eye comprises measuring a pH or near the surgical site.

7. The method of claim 1, wherein monitoring one or more surgical parameters within the eye comprises measuring a resistivity at or near the surgical site.

8. The method of claim 1, wherein monitoring one or more surgical parameters within the eye comprises measuring a conductivity at or near the surgical site.

9. The method of claim 1, wherein automatically adjusting one or more characteristics of the series of electrical pulses comprises adjusting at least one electrical parameter selected from the group consisting of:
a pulse frequency for at least one burst of the electrical pulses;
a pulse duty cycle for at least one burst of electrical pulses;
a burst repetition rate for two or more bursts of electrical pulses;
a pulse amplitude for one or more of the electrical pulses;
a pulse duration for one or more of the electrical pulses;
a pulse rise-time for one or more of the electrical pulses;
a pulse fall-time for one or more of the electrical pulses;
a pulse shape for one or more of the electrical pulses;
a pulse repetition rate for at least one burst of electrical pulses;
a duty cycle for two are more bursts of electrical pulses; and
the temporal relationship between pulses.

10. The method of claim 1, wherein the two or more electrodes of the HIPEF probe are configured to be selectively activated for each of the electrical pulses, and wherein automatically adjusting one or more characteristics of the series of electrical pulses comprises adjusting an activation sequence of the electrodes for at least one burst of the electrical pulses.

11. The method of claim 1, wherein automatically adjusting one or more characteristics of the series of electrical pulses is further based on an operator-selected surgical treatment type.

12. The method of claim 1, wherein automatically adjusting one or more characteristics of the series of electrical pulses is further based on an operator-selected surgical treatment location.

13. The method of claim 1, wherein automatically adjusting one or more characteristics of the series of electrical pulses is further based on an elapsed surgical treatment time.

14. The method of claim 1, wherein automatically adjusting one or more characteristics of the series of electrical pulses is further based on a cumulative metric indicative of energy delivered to the surgical site.

15. The method of claim 1, wherein automatically adjusting one or more characteristics of the series of electrical pulses comprises automatically adjusting electrical pulses and aspiration rate to achieve a desired flow rate.

16. The method of claim 1, wherein automatically adjusting one or more characteristics of the series of electrical pulses is further based on a probe distance from a retina.

17. An eye surgery apparatus, comprising:
a high-intensity pulsed-electrical-field (HIPEF) probe comprising at least two electrodes and configured for delivery of a high-intensity pulsed electrical field to a surgical site within an eye via the electrodes; and
at least one transducer configured to monitor one or more surgical parameters within the eye during application of the high-intensity pulsed electrical field to the surgical site;
a pulse generation circuit configured to generate a series of electrical pulses for application to the electrodes to create the high-intensity pulsed electrical field; and
a control circuit, operatively connected to the at least one transducer and the pulse generation circuit and configured to automatically adjust one or more characteristics of the series of electrical pulses, based on the one or more monitored surgical parameters.

18. The eye surgery apparatus of claim 17, wherein the at least one transducer comprises a flow rate sensor configured to measure aspiration flow rate.

19. The eye surgery apparatus of claim 17, wherein the at least one transducer comprises a pressure sensor configured to measure aspiration vacuum pressure.

20. The eye surgery apparatus of claim 17, wherein the at least one transducer comprises a bubble detection circuit configured to detect bubble formation at or near the surgical site.

21. The eye surgery apparatus of claim 17, wherein monitoring one or more surgical parameters within the eye comprises measuring a temperature at or near the surgical site.

22. The eye surgery apparatus of claim 17, wherein monitoring one or more surgical parameters within the eye comprises measuring a pH or near the surgical site.

23. The eye surgery apparatus of claim 17, wherein monitoring one or more surgical parameters within the eye comprises measuring a resistivity at or near the surgical site.

24. The eye surgery apparatus of claim 17, wherein monitoring one or more surgical parameters within the eye comprises measuring a conductivity at or near the surgical site.

25. The eye surgery apparatus of claim 17, wherein the control circuit is configured to automatically adjust one or more characteristics of the series of electrical pulses by providing a control signal to the pulse generation circuit, and wherein the pulse generation circuit is configured to selectively respond to the control signal by adjusting at least one electrical parameter selected from the group consisting of:
a pulse frequency for at least one burst of the electrical pulses;
a pulse duty cycle for at least one burst of electrical pulses;
a burst repetition rate for two or more bursts of electrical pulses;
a pulse amplitude for one or more of the electrical pulses;
a pulse duration for one or more of the electrical pulses;
a pulse rise-time for one or more of the electrical pulses;
a pulse fall-time for one or more of the electrical pulses;
a pulse shape for one or more of the electrical pulses;
a pulse repetition rate for at least one burst of electrical pulses;
a duty cycle for two are more bursts of electrical pulses; and
the temporal relationship between pulses

26. The eye surgery apparatus of claim 17, wherein the two or more electrodes of the HIPEF probe are configured to be selectively activated by the pulse generator circuit for each of the electrical pulses, and wherein the control circuit is configured to automatically adjust one or more characteristics of the series of electrical pulses by providing a control signal to the pulse generation circuit directing the pulse generation circuit to adjust an activation sequence of the electrodes for at least one burst of the electrical pulses.

27. The eye surgery apparatus of claim 17, wherein the control circuit is configured to automatically adjust one or more characteristics of the series of electrical pulses based further on an operator-selected surgical treatment type.

28. The eye surgery apparatus of claim 17, wherein the control circuit is configured to automatically adjust one or more characteristics of the series of electrical pulses based further on an operator-selected surgical treatment location.

29. The eye surgery apparatus of claim 17, wherein the control circuit is configured to automatically adjust one or more characteristics of the series of electrical pulses based further on an elapsed surgical treatment time.

30. The eye surgery apparatus of claim 17, wherein the control circuit is configured to automatically adjust one or more characteristics of the series of electrical pulses based further on a cumulative metric indicative of energy delivered to the surgical site.

31. The eye surgery apparatus of claim 17, wherein the control circuit is configured to automatically adjust one or more characteristics of the series of electrical pulses comprises automatically adjusting electrical pulses and aspiration rate to achieve a desired flow rate.

32. The eye surgery apparatus of claim 17, wherein the control circuit is configured to automatically adjust one or more characteristics of the series of electrical pulses is further based on a probe distance from a retina.
